(19) United States
(12) Patent Application Publication
(10) Pub. No.: US 2024/0057990 A1
(43) Pub. Date: Feb. 22, 2024

(54) SUTURE ANCHOR

(71) Applicant: Anika Therapeutics, Inc., Bedford, MA (US)

(72) Inventors: Greg Denham, Lakewood Ranch, FL (US); Joel Harshbarger, Palmetto, FL (US); Adrian Bosworth, Bradenton, FL (US)

(21) Appl. No.: 17/892,796

(22) Filed: Aug. 22, 2022

(51) Int. Cl. A61B 17/04

(52) U.S. Cl. CPC A61B 17/0401; A61B 2017/0409; A61B 2017/0414; A61B 2017/0412

(57) ABSTRACT

A suture anchoring system includes a suture anchor and an anchor insertion device. The suture anchor has an elongated anchor body having proximal and distal ends and a long axis, with an elongated axial drive opening extending along the long axis. A plurality of axial drive grooves has a long axis parallel to the long axis of the elongated anchor body, a radially inward and axially extending groove opening communicating with the axial drive opening, a contact surface radially outward from the groove opening, and side surfaces connecting the contact surface and the groove opening. The radially outward contact surface has a greater width than that of the groove opening. Threads are provided on the outside surface of the anchor body. The drive shaft has radially outwardly extending axial drive projections configured to mate with the axial drive grooves.

### FIELD OF THE INVENTION

The present invention relates generally to suture anchors, and more particularly suture anchors that are capable of being used in both a knotted and knotless configuration.

### BACKGROUND OF THE INVENTION

Suture anchors are used to attach tendons, ligaments, soft tissue and implants such as synthetic grafts, to bone. The surgeon proceeds by fashioning a tunnel in the bone and inserting an anchor into the tunnel which will secure a suture to the bone. The suture anchor can have external threads in the manner of a screw such that when rotated the suture anchor will advance securely into the tunnel and resist removal.

The suture anchor is mounted to an anchor insertion device having a handle and a drive shaft with structure for engaging and retaining the suture anchor. The suture anchor has a socket for engaging a mating head portion of the drive shaft so as to secure the suture anchor to the drive shaft and allow for the rotation of the suture anchor by manipulation of the insertion device so as to thread the suture anchor into the tunnel. The insertion device is used to position the suture anchor at the tunnel formed in the bone, and then the drive shaft is rotated so as to thread the suture anchor into the bone tunnel. Any knotless sutures required for the repair are passed thought the distal eyelet in the anchor tip and are then passed through the tissue prior to anchor insertion. The sutures are tensioned to approximate the tissue to the bone and become locked in place between the threads of the anchor and the bone tunnel after the anchor is inserted. Any sliding sutures are passed through the tissue after insertion, tensioned and knots are tied to secure the tissue in place. The drive shaft is removed from the socket of the suture anchor, the repair is completed, and the incision is closed.

The strength and integrity of the suture anchor is of paramount importance to ensure an efficient and long-lasting repair. In the past, such suture anchors were made from implantable metals or alloys which had high strength and durability, but were difficult and costly to fashion. More recently, polymer, ceramic, or composite materials have been developed. These include biodegradable and nonbiodegradable materials. Such materials are less expensive and easier to manufacture than their metal counterparts.

The suture anchor must be able to withstand the forces and particularly the torque that is necessary to properly insert the suture anchor into the tunnel. Failure of the suture anchor in this regard can result in stripping of the socket or even shearing of a portion of the suture anchor. There is a need for suture anchors that are easily manufactured, are capable of withstanding necessary torque forces during insertion, and will also provide a durable and long-lasting repair.

### SUMMARY OF THE INVENTION

A suture anchoring system includes an anchor insertion device comprising a handle and a drive shaft with a long axis. The suture anchor has an elongated anchor body having proximal and distal ends and a long axis, with an elongated axial drive opening extending along the long axis. The axial drive opening defines an inside surface of the anchor body. The anchor body has an outside surface radially outward from the inside surface. A plurality of axial drive grooves have a long axis parallel to the long axis of the elongated anchor body. The drive grooves have a radially inward and axially extending groove opening communicating with the axial drive opening, and a contact surface radially outward from the groove opening. The radially outward contact surface has a greater width than the groove opening. The axial drive grooves further include side surfaces extending between the contact surface and the axially extending opening of the axial drive grooves. The side surfaces can be oriented at an acute angle to the radially outward contact surface. Threads are provided on the outside surface of the anchor body for engaging the bone. The drive shaft of the anchor insertion device comprises radially outwardly extending axial drive projections configured to mate with the axial drive grooves.

The axial drive grooves can be circumferentially spaced apart and equidistant from adjacent axial drive grooves. The axial drive projections can be similarly circumferentially spaced apart and equidistant from adjacent axial drive projections, such that the drive shaft and the axial drive projections can be axially inserted into the elongated axial drive opening and the axial drive grooves.

The threads are provided as a double helix or other multi-start thread configuration. The threads can be buttress style threads or have a thread profile which has a sloping leading edge angle to easily displace the bone during insertion and a trailing edge angle that is less sloping to better resist the tensile forces of the sutures pulling on the anchor and prevent loss of fixation in the bone

The axial drive grooves in a lateral cross section orthogonal to the long axis of the anchor body can be isosceles trapezoidal in shape. The axial drive projections of the drive shaft of the anchor insertion device can, in a lateral cross section orthogonal to the long axis of the drive shaft, also be isosceles trapezoidal in shape so as to mate with and engage the axial drive grooves. The contact surface can be at least one selected from the group consisting of planar and curved. The contact surface can be concave.

The axial drive opening and the axial drive grooves can extend from the proximal end to the distal end of the elongated anchor body. Friction projections can be provided on the inside surface of the axial drive opening to assist in retaining the suture anchor in position prior to deployment.

The anchor body can further comprise of a plurality of flow passages for permitting ingress of biological material from a patient into the axial drive opening. The anchor body can include elongated radially inwardly extending braces between the axial drive grooves. The plurality of flow passages can be positioned so as to pass through the braces.

The anchor body can comprise biocompatible plastic, the biocompatible plastics comprising at least one selected from the group of biocomposite and nonbiocomposite materials. One such biocomposite material is poly(L-lactide-co-glycolide) and β-TCP in a 70:30 ratio, and one such nonbiocomposite plastic material is polyether ether ketone (PEEK).

The suture anchor can include a suture retention tip comprising at least one eyelet for receiving a suture. The suture retention tip further can have an open interior and a retention post in the open interior for engaging a retention suture. The suture retention tip can be detachable from the anchor body.

A suture anchor includes an elongated anchor body having proximal and distal ends and a long axis, with an elongated axial drive opening extending along the long axis. The axial drive opening defines an inside surface of the anchor body. The anchor body has an outside surface radially outward from the inside surface. A plurality of axial drive grooves has a long axis parallel to the long axis of the elongated anchor body. The drive grooves have a radially inward and axially extending groove opening communicating with the axial drive opening, and a contact surface radially outward from the groove opening. The radially outward contact surface has a greater width than the groove opening. The axial drive grooves further comprise side surfaces extending between the contact surface and the axially extending opening of the axial drive grooves. The side surfaces are oriented at an acute angle to the radially outward contact surface. Threads are provided on the outside surface of the anchor body.

The axial drive grooves can be circumferentially spaced apart and equidistant from adjacent axial drive grooves. The axial drive grooves, in a lateral cross section orthogonal to the long axis, can be isosceles trapezoidal in shape.

The threads can be provided as a double helix. The threads can be buttress threads.

A suture anchor having a suture anchor body having an external surface, can have a double helix thread on the external surface. The double helix thread can be a buttress thread. The suture anchor can be made from a plastic material. The suture anchor can comprise an open interior, and plurality of flow passages in the suture anchor body communicating with the open interior.

### BRIEF DESCRIPTION OF THE DRAWINGS

There are shown in the drawings embodiments that are presently preferred it being understood that the invention is not limited to the arrangements and instrumentalities shown, wherein:

FIG. 1 is a top perspective view of a suture anchor according to the invention.

FIG. 2 is a bottom perspective view.

FIG. 3 is a front elevation.

FIG. 4 is a rear elevation.

FIG. 5 is a top plan view.

FIG. 6 is a bottom view.

FIG. 7 is an enlarged view of area A7 in FIG. 5.

FIG. 8 is a cross-section taken along line 8-8 in FIG. 5.

FIG. 9 is a top perspective view of a cross-section taken along line 9-9 in FIG. 5.

FIG. 10 is a schematic depiction of a suture anchor according to the invention, partially in phantom to depict double helix threads.

FIG. 11 is a top plan view of the schematic depiction of FIG. 10.

FIG. 12 is an enlarged view of area A12 in FIG. 8.

FIG. 13 is a perspective view of a suture anchoring system according to the invention.

FIG. 14 is an enlarged view of area A14 in FIG. 13.

FIG. 15 is a side elevation.

FIG. 16 is an enlarged view of area A16 in FIG. 15

FIG. 17 is a cross-section taken along line 17-17 in FIG. 16.

FIG. 18 is a cross-section taken along line 18-18 in FIG. 16.

FIG. 19 is perspective view of the suture anchoring system partially disassembled.

FIG. 20 is an enlarged view of area A20 in FIG. 19.

FIG. 21 is a perspective view, partially broken away, of a drive shaft and anchor tip in a first stage of assembly.

FIG. 22 is a perspective view, partially broken away, of the drive shaft and anchor tip in a subsequent stage of assembly.

FIG. 23 is a cross-section taken along line 23-23 in FIG. 21.

FIG. 24 is a cross-section taken along line 24-24 in FIG. 22.

FIG. 25 is a perspective view of a drive shaft, suture anchor and anchor tip.

FIG. 26 is a cross-section of a suture anchoring system, in a first mode of operation.

FIG. 27 is an enlarged view of area A27 in FIG. 26.

FIG. 28 is a cross-section of the suture anchoring system, in a second mode of operation.

FIG. 29 is an enlarged view of area A29 in FIG. 28.

FIG. 30 is a perspective view of the suture anchoring system in the second mode of operation.

FIG. 31 is an enlarged view of area A31 in FIG. 30.

FIG. 32 is a perspective view of the suture anchoring system in a third mode of operation.

FIG. 33 is an enlarged view of area A33 in FIG. 32.

FIG. 34 is a schematic illustration, partially broken away and partially in cross-section, of an example of a first step of a method for anchoring a suture using the suture anchoring system of the invention.

FIG. 35 is a cross-section of a second step.

FIG. 36 is a side elevation of a suture anchoring system, partially broken away, illustrating a third step.

FIG. 37 is a side elevation, partially in cross-section, of a fourth step.

FIG. 38 is a side elevation, partially broken away and partially in cross-section, of a fifth step.

FIG. 39 is a side elevation, partially broken away and partially in cross-section, of a sixth step.

FIG. 40 is a side elevation, partially broken away and partially in cross-section, of a seventh step.

FIG. 41 is a side elevation, partially broken away and partially in cross-section, of the seventh step of FIG. 40.

FIG. 42 is a side elevation, partially broken away and partially in cross-section, of an eighth step.

FIG. 43 is a side elevation, partially broken away and partially in cross-section, of a ninth step.

FIG. 44 is an enlarged side elevation, partially broken away and partially in cross-section, of the ninth step of FIG. 43.

FIG. 45 is an enlarged cross-section, partially broken away and partially in cross-section, of a tenth step.

FIG. 46 is an enlarged cross-section, partially broken away and partially in cross-section, of an eleventh step.

FIG. 47 is an enlarged cross-section, partially broken away and partially in cross-section, illustrating an alternative method.

FIG. 48 is an enlarged cross section of the alternative method of FIG. 47, in a subsequent stage.

### DETAILED DESCRIPTION OF THE INVENTION

A suture anchoring system includes a suture anchor and an anchor insertion device comprising a handle and a drive shaft with a long axis. The suture anchor has an elongated anchor body having proximal and distal ends and a long axis, with an elongated axial drive opening extending along the long axis. The axial drive opening defines an inside surface of the anchor body, and the anchor body has an outside surface radially outward from the inside surface. A plurality of axial drive grooves has a long axis parallel to the long axis of the elongated anchor body. The drive grooves have a radially inward and axially extending groove opening communicating with the axial drive opening, and a contact surface radially outward from the groove opening. The radially outward contact surface has a greater width than that of the groove opening. The axial drive grooves further include side surfaces extending between the contact surface and the axially extending opening of the axial drive grooves. The side surfaces are oriented at an acute angle to the radially outward contact surface and can have the same dimension. Threads are provided on the outside surface of the anchor body. The drive shaft of the anchor insertion device has radially outwardly extending axial drive projections configured to mate with the axial drive grooves.

The axial drive grooves can be circumferentially spaced apart and equidistant from adjacent axial drive grooves. The axial drive projections on the drive shaft are similarly circumferentially spaced apart and equidistant from adjacent axial drive projections, such that the drive shaft and the axial drive projections can be axially inserted into the elongated axial drive opening and the axial drive grooves. Four such drive groves can be provided and circumferentially spaced apart, however more or fewer of such drive grooves are possible.

The threads of the suture anchor can have differing thread characteristics such as dimensions, thread shape and pitch. The threads can be provided as a double helix or in another multi-start thread configuration. Double helix threads advance the suture anchor further with each rotation, such that fewer rotations will be necessary to advance the suture anchor into position. The threads can be buttress threads. Buttress threads present an asymmetric thread profile, with the distal face being slanted and the proximal face perpendicular to the long axis, or less slanted than the distal face. The more perpendicular proximal face will resist pull out upon the application of an axial load. A suture anchor with such double helix or other multi-start helical threads can have differing configurations, and can be made with a drive opening that extends axially through the anchor body, or only partially extends into the anchor body in the form of a socket for the drive head of a drive shaft.

The precise shape of the axial drive grooves can vary. For example, in a lateral cross section orthogonal to the long axis of the anchor body, the axial drive grooves can be isosceles trapezoidal in shape, and in a lateral cross section orthogonal to the long axis of the drive shaft, the axial drive projections can be isosceles trapezoidal in shape. As used herein the term isosceles trapezoidal is intended to include sides that may be slightly nonplanar so long as imaginary lines connecting the endpoints of the sides result in a configuration of an isosceles trapezoid. For example, the contact surface can be either planar or curved. The contact surface can be concave and the drive projections on the drive shaft can have a matching convex surface.

The axial drive opening and the drive grooves form a socket to engage the projections of the anchor insertion device. The axial drive grooves can extend from the proximal end to the distal end of the elongated anchor body. In this manner, the suture anchor can slide onto the projections of the drive shaft of the anchor insertion device and will be retained on the drive shaft until it is discharged by operation of the anchor insertion device. The suture anchor can further include friction projections on the inside surface of the axial drive opening to help to retain the anchor until it is ready to be released from the anchor insertion device. It is also possible that the drive opening and the drive grooves extend only partially from the proximal end of the anchor body, forming a socket for the drive shaft and projections at the distal end of the anchor insertion device.

The anchor body can have a plurality of flow passages. The flow passages can allow for egress of certain materials, such as Tactoset® (Anika Therapeutics, Inc., Bedford, MA) bone void substitute that can be injected through the anchor to encourage bone ingrowth and augment the fixation of the suture anchor. The anchor body can have elongated radially inwardly extending braces between the axial drive grooves. The flow passages can pass through the braces such that the anchor body will be reinforced at the location where the flow passages are present.

The suture anchor can be made from a variety of different materials. The anchor body can comprise biocompatible plastic. The biocompatible plastics can include at least one selected from the group of biocomposite and non-biocomposite plastics. One such biocomposite material is poly(L-lactide-co-glycolide) and β-TCP in a 70:30 ratio, and one such non-biocomposite material is polyether ether ketone (PEEK). Other materials are possible.

The suture anchor can further include a suture anchor tip that has a pointed end for displacing biological material as it is inserted into position. The suture anchor tip can be detachable from the anchor body. The suture anchor tip can have structure such as a lateral eyelet or axial opening for passing a suture through the suture anchor tip. The suture anchor tip can have an open interior and a retention post in the open interior for engaging the suture. The suture can be part of the repair, or can be a retention suture that is used to retain the detachable suture anchor tip until it is ready to be deployed.

A method for securing a suture includes the step of providing the suture anchor system including a suture anchor as described and an anchor insertion device with a drive shaft for engaging the suture anchor. The suture anchor can be pre-loaded onto the drive shaft and the assembly packaged in a sterile packaging for ready use. The method includes the step of engaging the drive shaft to the suture anchor with the axial drive projections positioned in and engaging the axial drive grooves. The suture anchor engaged to the drive shaft is positioned in a target location. The drive shaft is rotated and the suture anchor will also rotate. A force is applied by the anchor insertion device while advancing the suture anchor such that the threads engage the bone or tissue surrounding the target location. The suture anchor system can further include a suture anchor tip, and the method can include the step of positioning the suture anchor tip on the shaft and securing it with a tip retention suture. The threads on the suture anchor can be provided as a double helix, and the double helix will advance the suture anchor more rapidly with turns of the handle.

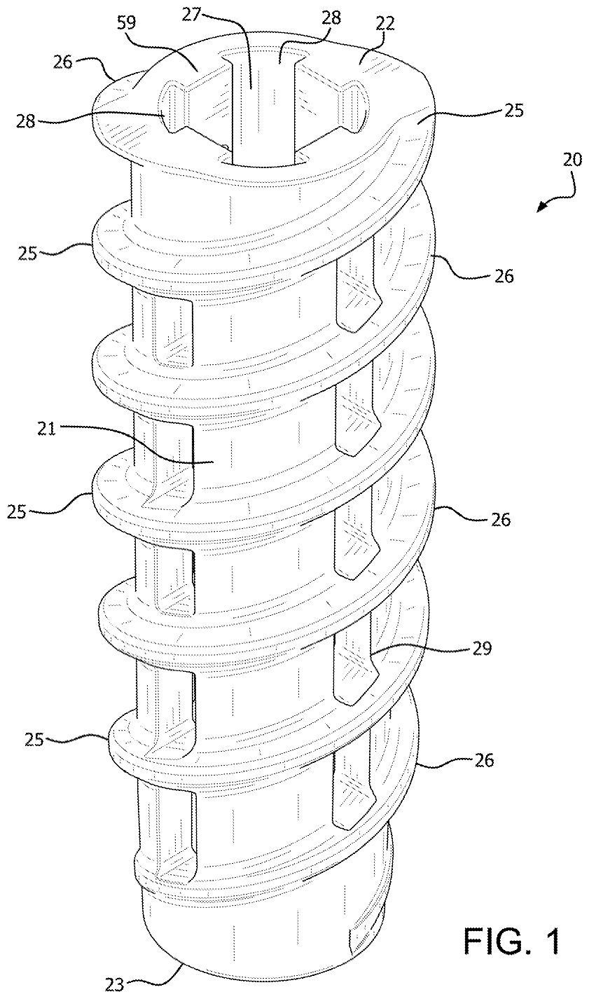

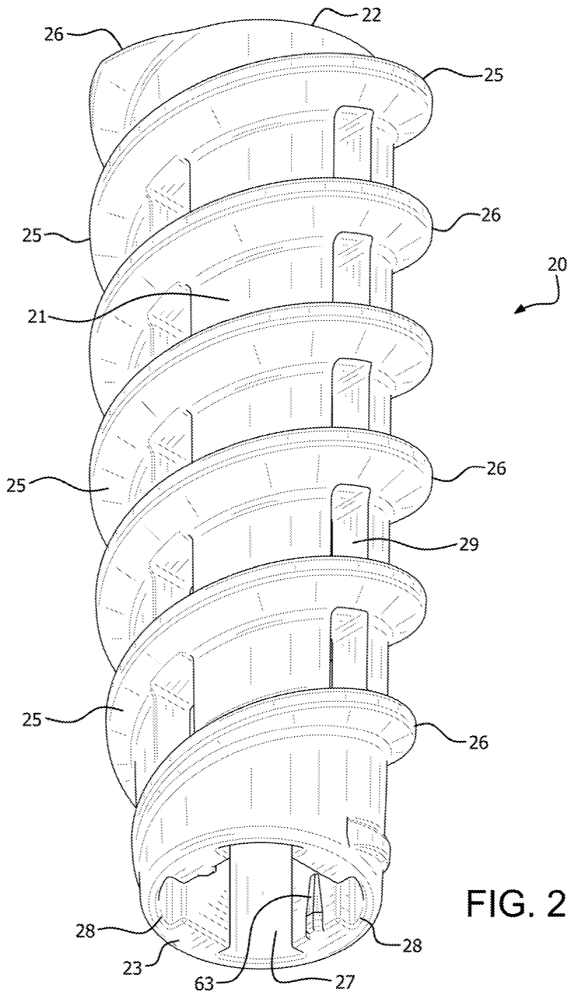

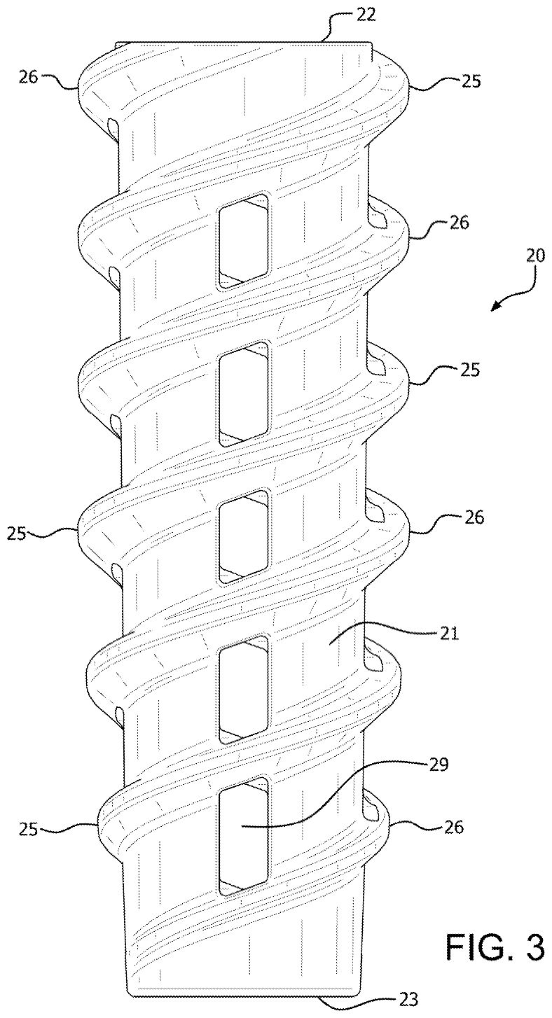

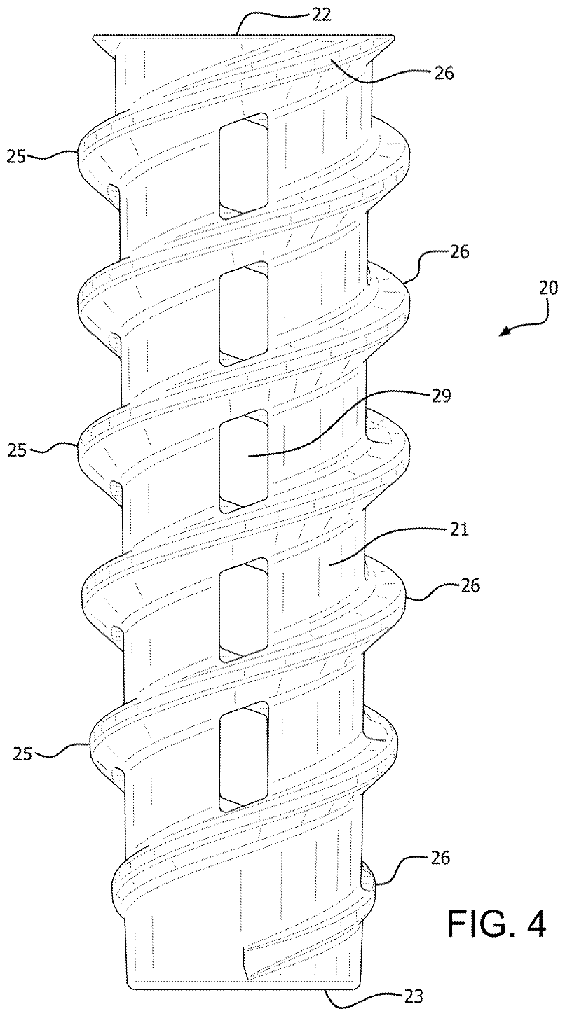

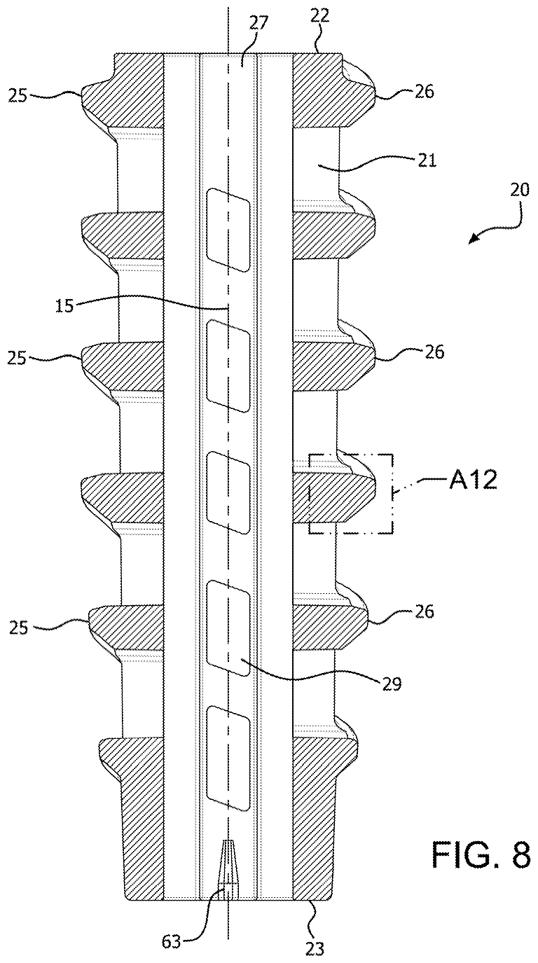

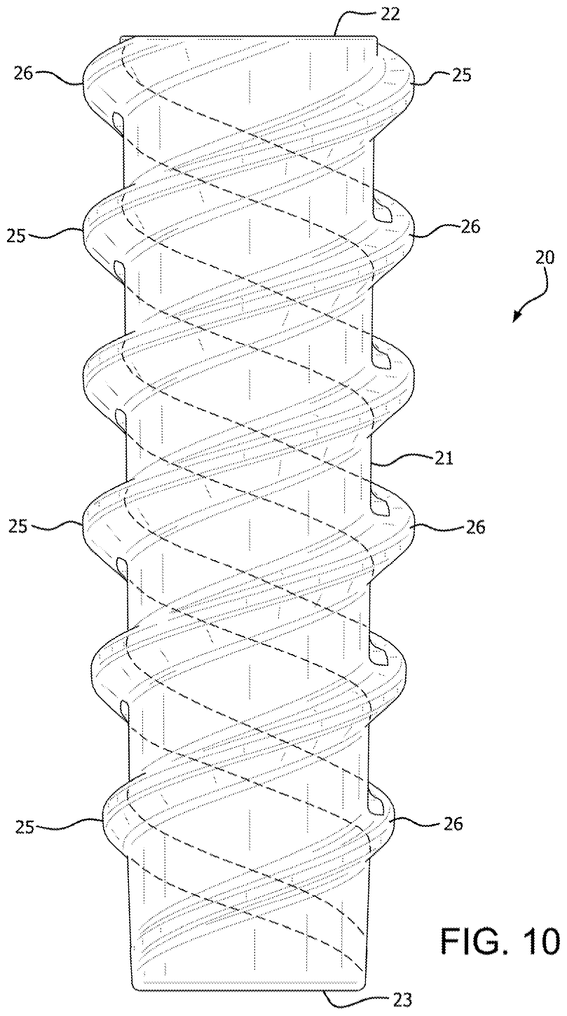

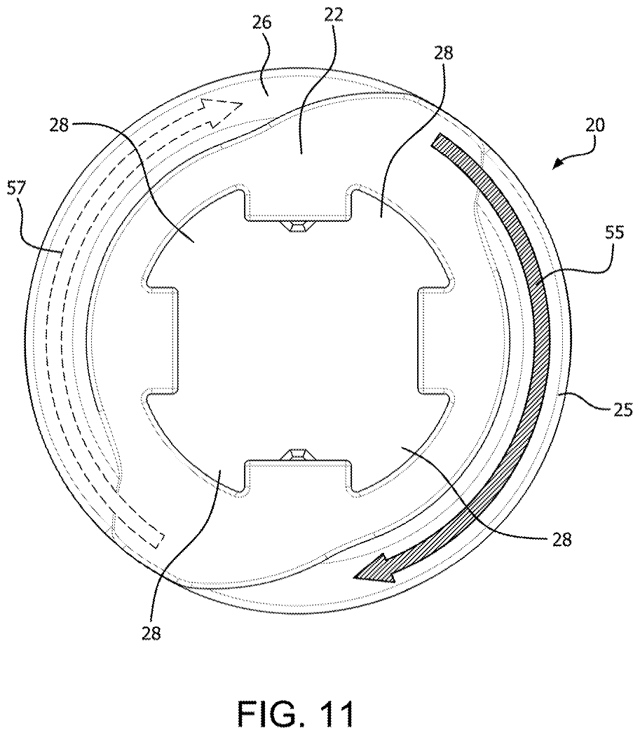

There is shown in FIGS. 1-12 a suture anchor 20 according to the invention. The suture anchor 20 includes an anchor body 21 with a proximal end 22 and distal end 23. The suture anchor 20 can have a first helical thread 25 located on an exterior surface of the anchor body 21 for purposes of engaging a bone tunnel. In the embodiment shown, the suture anchor 20 has a second helical thread 26 interwoven with the first helical thread 25 to facilitate advancement of the suture anchor 20 in a bone tunnel. The invention can also be utilized with a single helical thread, or with more helical threads. The orientation of the helical threads 25 and 26 is shown particularly in FIG. 10, where phantom lines represent the helical threads on an opposing side of the anchor body 21. As shown in FIG. 11 by the arrows 55 for the first helical thread 25, and arrow 57 for the second helical thread 26, the first helical thread 25 and second helical thread 26 progress from the proximal end 22 to the distal and 23 in the same circumferential direction and begin at opposite radial locations on the anchor body 21.

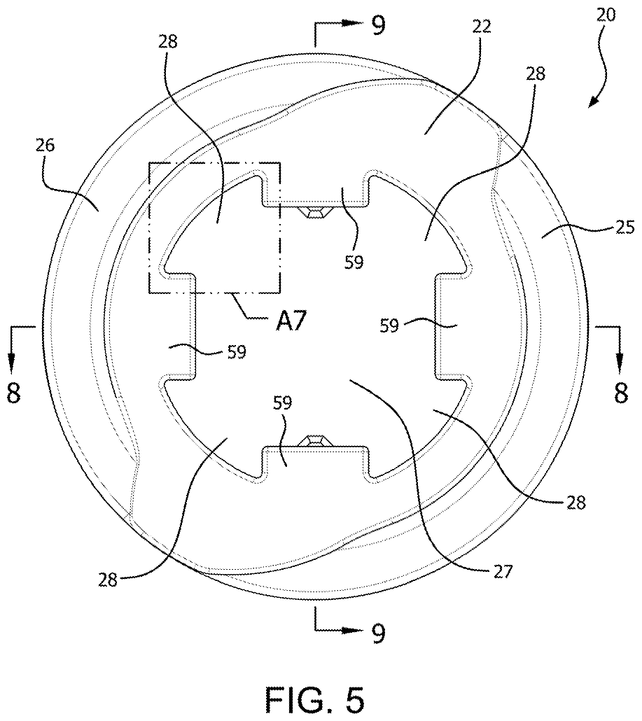

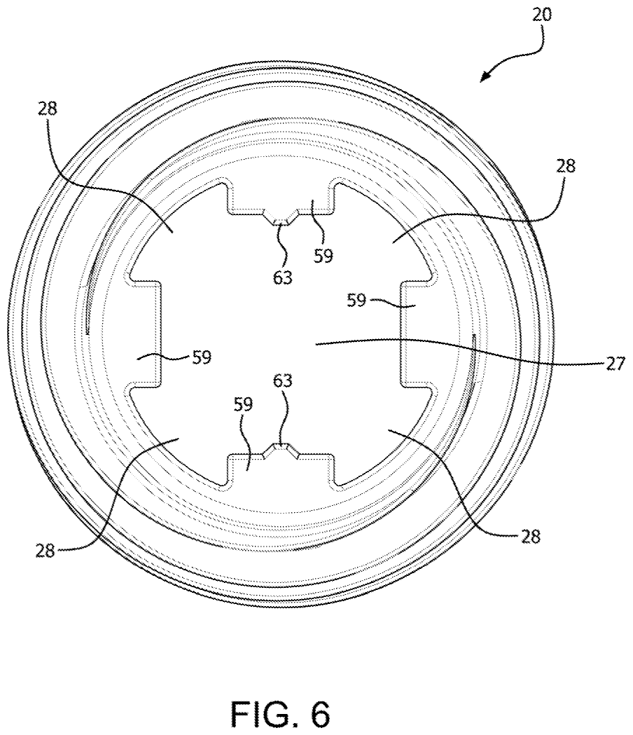

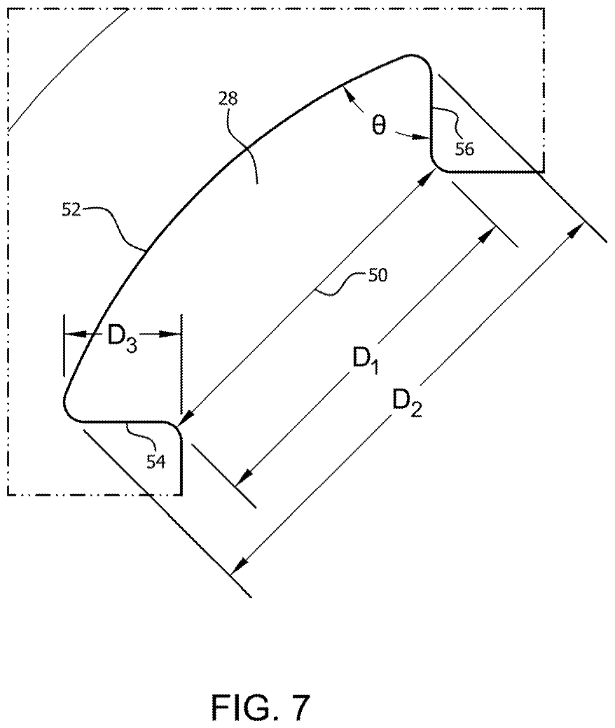

The anchor body 21 has an interior drive opening 27 with a long axis 15 which forms a socket for receiving the distal end drive head of a drive shaft for purposes of rotating the anchor 20 such that the helical threads 25 and 26 of the anchor 20 will engage the bone tunnel and advance the suture anchor 20 into the bone tunnel. A plurality of axial drive grooves 28 have a long axis 17 parallel to the long axis 15 of the elongated anchor body (FIGS. 5-6). The drive grooves 28 have a radially inward and axially extending groove opening indicated by arrow 50 (FIG. 7) communicating with the axial drive opening 27, and a contact surface 52 radially outward from the groove opening 50. The radially outward contact surface 52 has a greater width D₂ than the width D₁ of the groove opening 50. The axial drive grooves 28 further comprise side surfaces 54 and 56 extending between the contact surface 52 and the axially extending opening 50 of the axial drive grooves 28. The side surfaces 54 and 56 can be oriented at an acute angle θ to the radially outward contact surface 52. It can be seen the axial drive grooves 28 in lateral cross section can be in general form isosceles trapezoidal in shape. Parallel sides are formed by the groove opening 50 and the contact surface 52, and non-parallel sides 54 and 56 between the groove opening 50 and the contact surface 52 can be of equal length D₃. The length D₃ can be less than the length D₂ of the contact surface 52, and can also be less than the length D₁ of the groove opening 50. The contact surface 52 and sides 54 and 56 can be planar as shown for the sides 54 and 56, or slightly curved as shown for the contact surface 52 which is in the depicted embodiment concave.

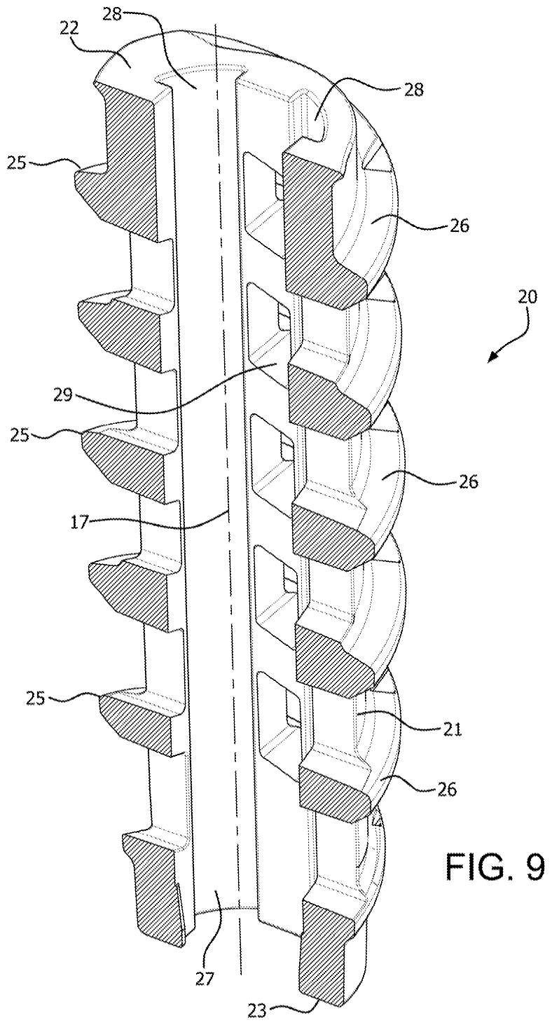

The suture anchor 20 can also include a plurality of flow passages 29 which are openings in the anchor body 21 communicating between the exterior and the interior opening 27. The flow passages 29 are positioned between the helical threads 25 and 26 and after implantation permit the ingress of bodily fluid and tissue into the interior drive opening 27 so as to integrate the suture anchor 20 with surrounding soft tissue and bone. Braces 59 can be provided between the axial drive grooves 28 and are thicker wall portions of the anchor body 21 than the wall portions radially outward from the drive grooves 28, as shown particularly in FIG. 9. The provision of the flow passages 29 in these braces provides added rigidity to this area despite the presence of the openings of the flow passages 29. Any number and orientation of flow passages 29 can be provided.

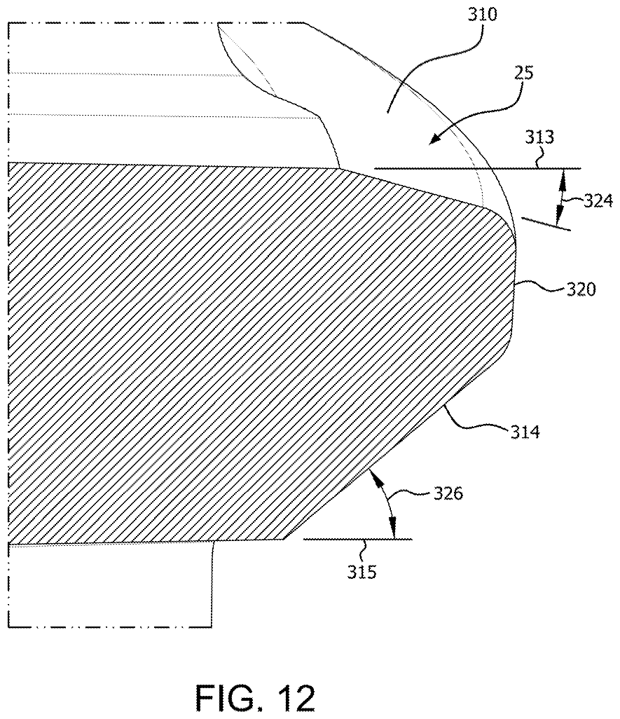

The helical threads 25 and 26 can take differing forms, including dimensions, thread shape and pitch. There is shown in FIG. 12 the helical thread 25 provided as a buttress thread. The buttress thread has a proximal surface 310, a distal surface 314, and an outer surface 320. The angle 324 of the distal surface 310 to a perpendicular 313 of the long axis 15 of the anchor 20 is less than the angle 326 of the distal surface 314 to a perpendicular 315 to the long axis 15 of the anchor 20. In this manner the more slanted distal surface 314 will facilitate advancement of the thread 25 and the anchor 20 into a bone tunnel, and the more perpendicular surface 310 will resist removal of the anchor 20 from the bone tunnel. Other thread shapes are possible.

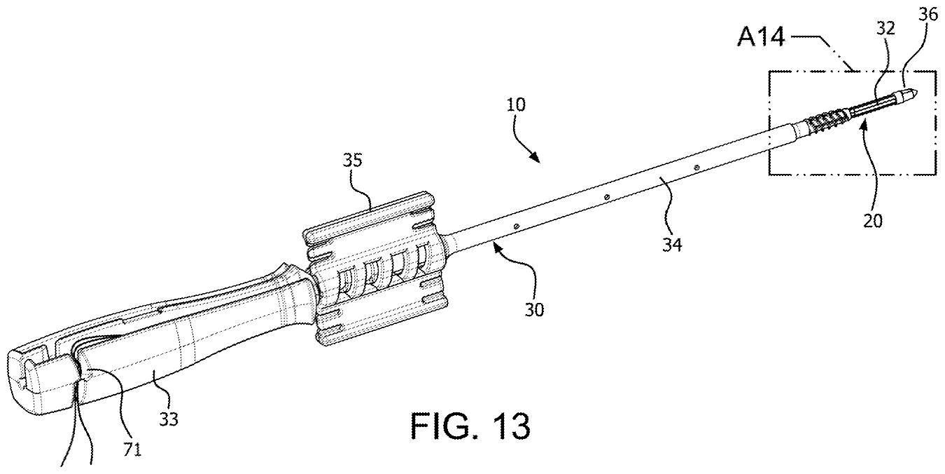

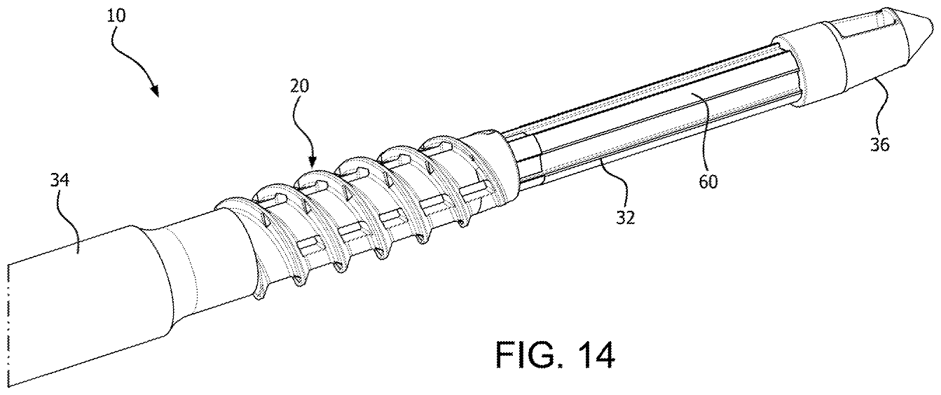

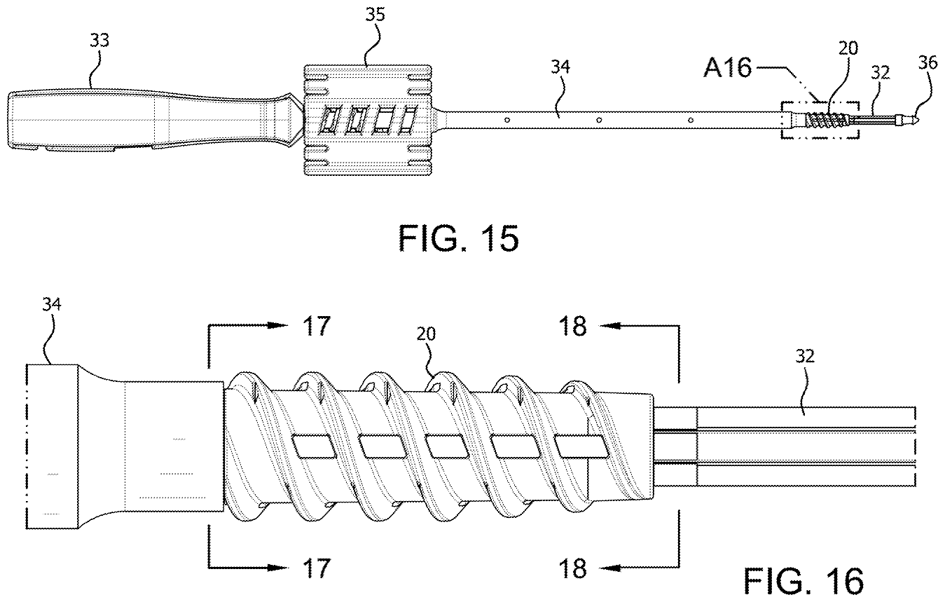

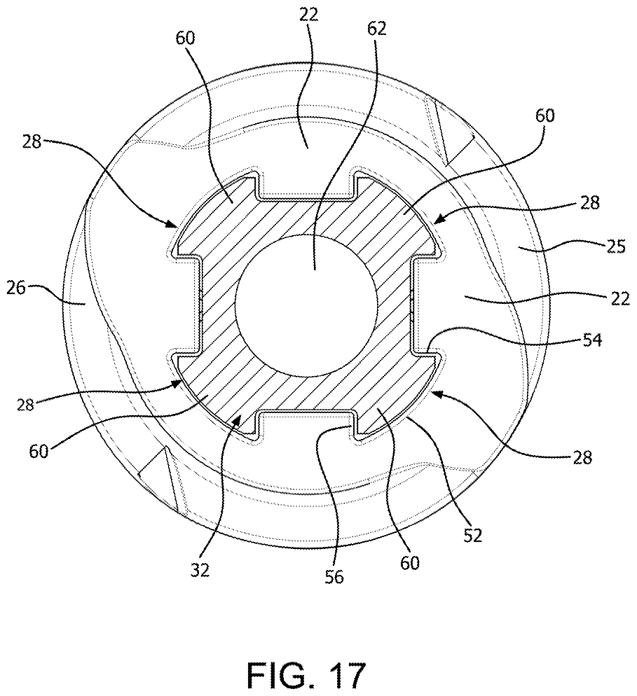

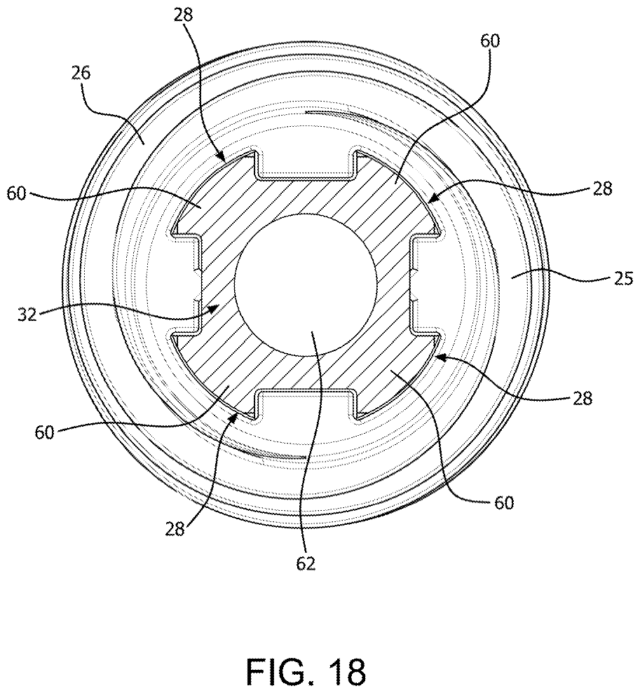

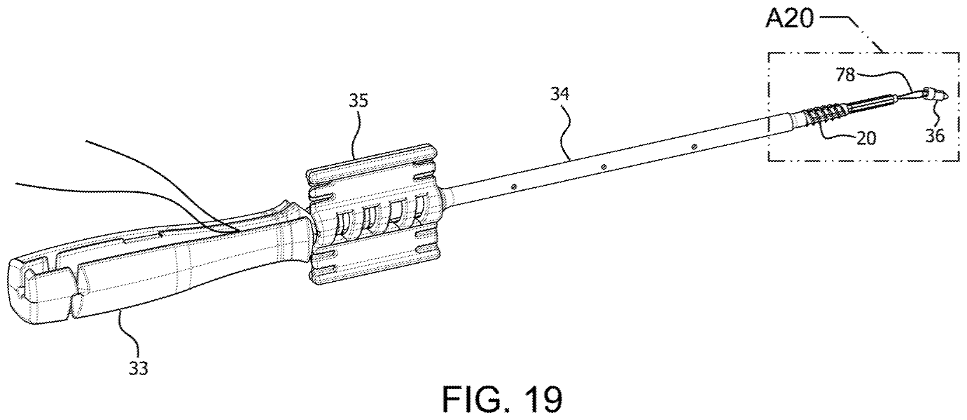

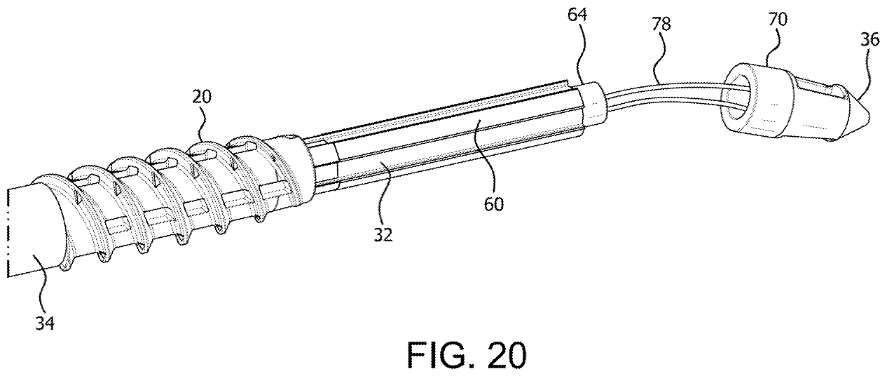

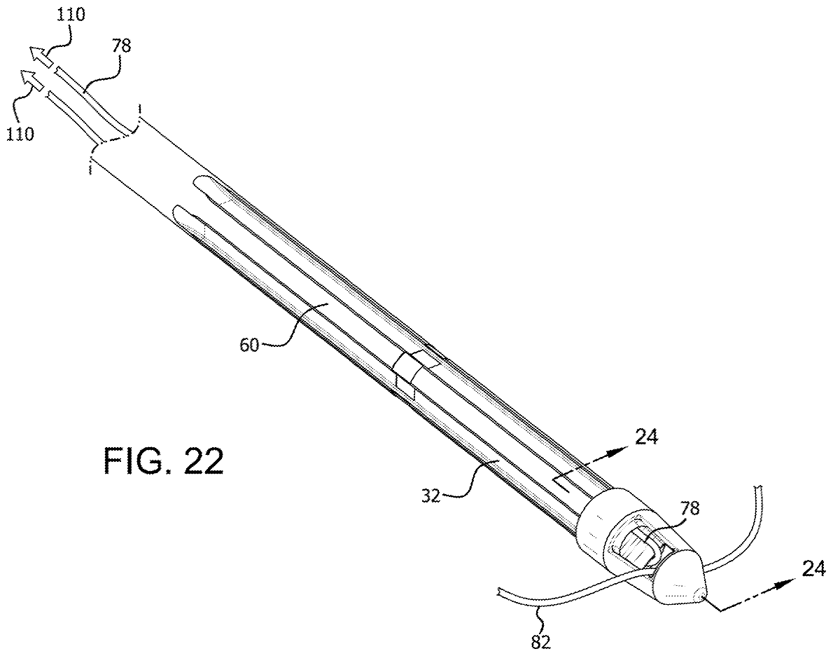

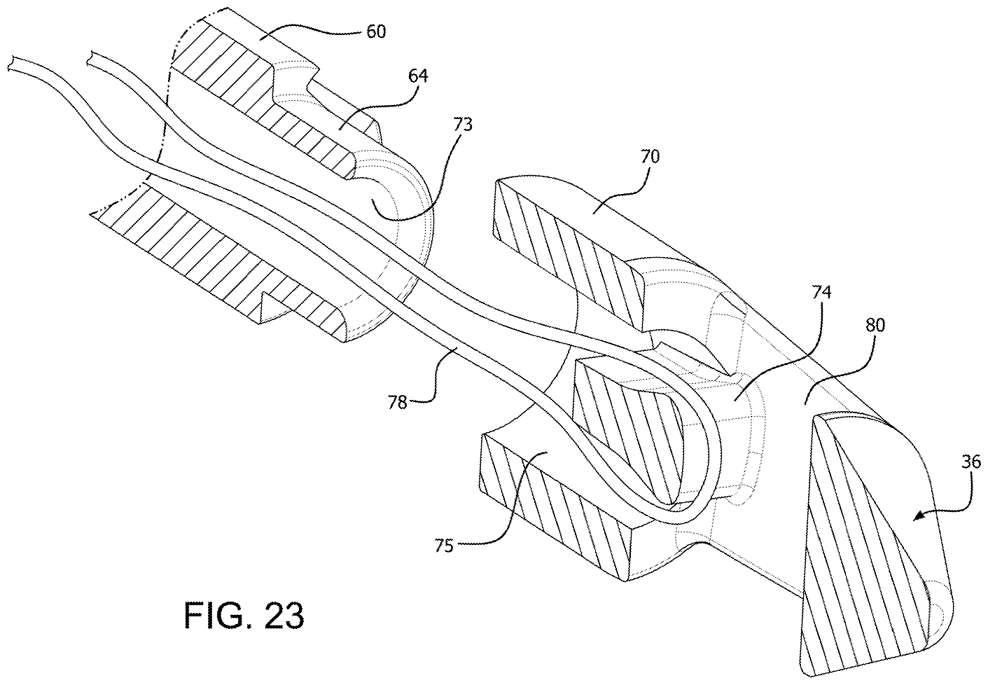

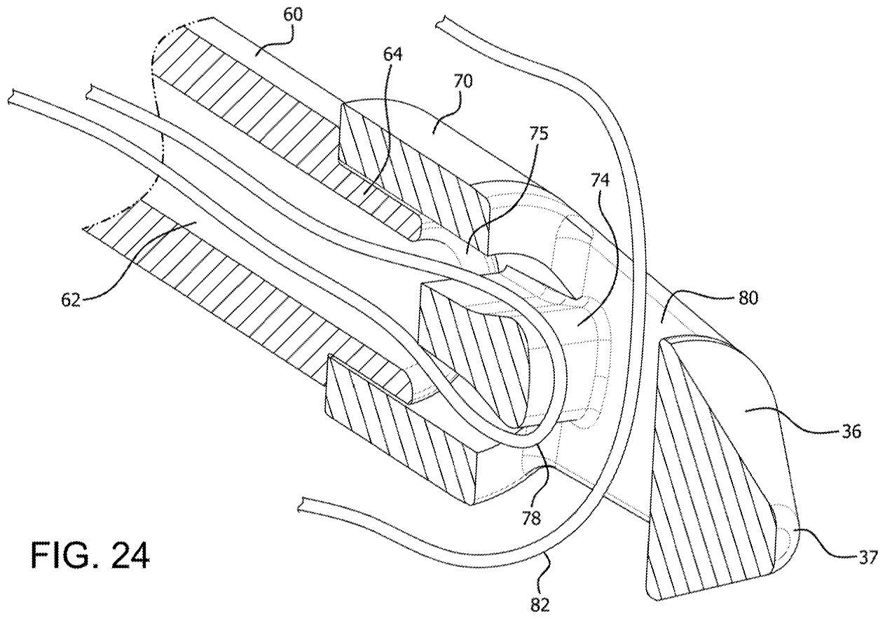

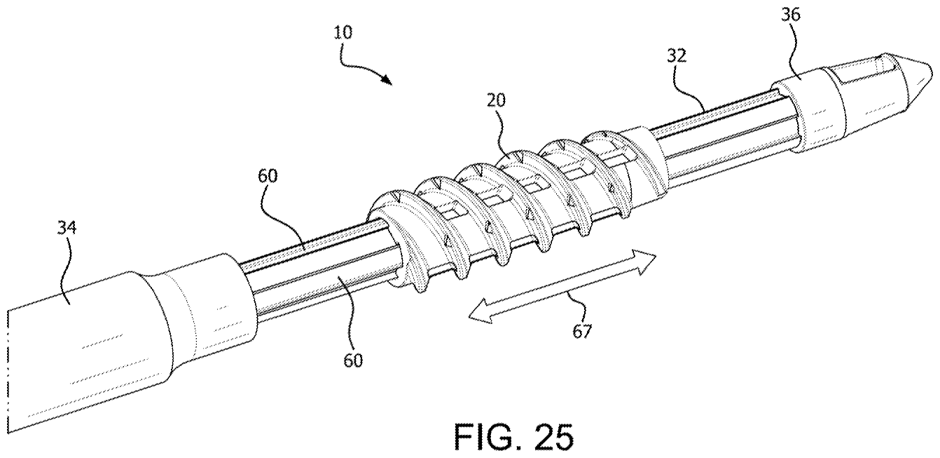

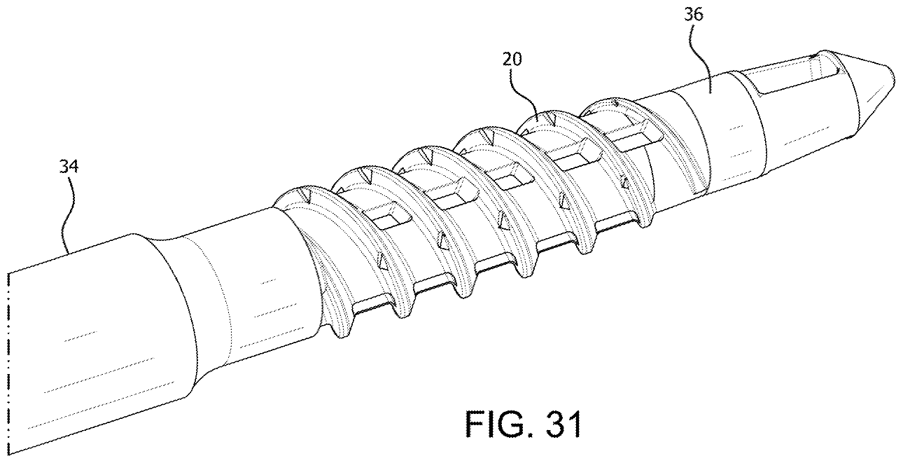

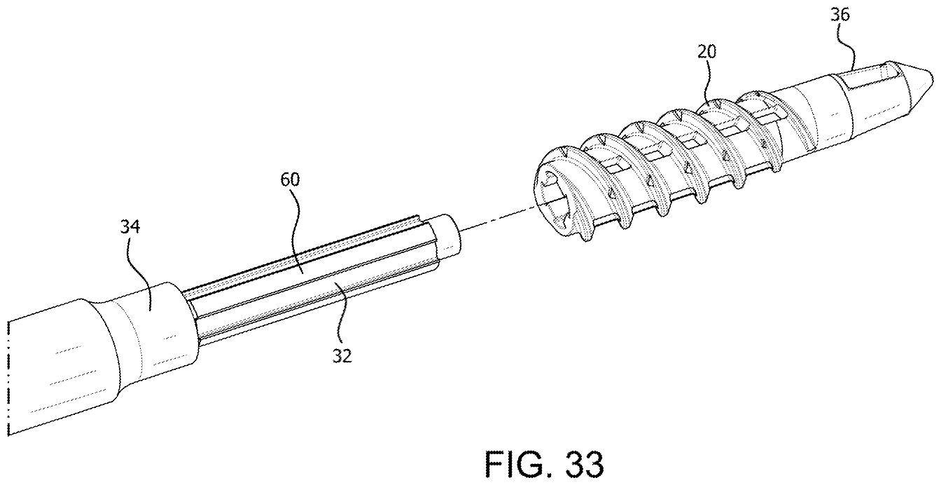

There is shown in FIGS. 13-33 a suture anchoring system 10 including the suture anchor 20 and an anchor insertion device 30. The anchor insertion device 30 includes a drive shaft 32, a handle 33, and a drive paddle sheath 34. The drive paddle sheath 34 communicates with a drive paddle 35. A suture anchor tip 36 with a pointed distal end 37 can be secured to a distal end of the drive shaft 32. As shown in FIGS. 17-18, the drive shaft 32 includes axially extending drive projections 60 which are configured to engage the axially extending drive grooves 28. The drive projections 60 can be isosceles trapezoidal in shape to mate with and engage the drive grooves 28. The anchor 20 can be slidably engaged to the drive shaft 32 by positioning the distal end of the drive shaft 32 to the proximal and of the anchor 20 and sliding the suture anchor 20 onto the drive shaft 32 and then positioning the suture anchor tip 36 at the distal end of the drive shaft 32 (FIG. 22). The anchor 20 can slide in either direction on the drive shaft 32, as shown by the arrow 67 is FIG. 25. The drive shaft can have an open interior 62 to accept a retention suture or fixation suture as will be described. Friction projections 63 can be provided at the distal end 23 of the suture anchor 20, and can extend radially inward into the open interior 62 to frictionally engage the drive shaft 32 and maintain the suture anchor 20 in position.

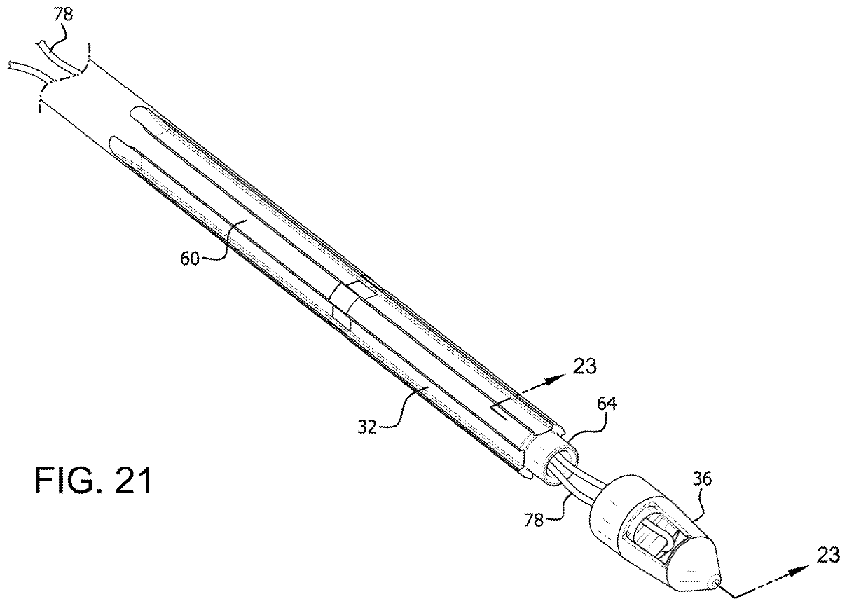

As shown in FIGS. 21-24, the drive shaft 32 can have a neck 64 which can engage a collar portion 70 of the suture anchor tip 36. A post 74 can be provided in an open interior 75 of the collar portion 70 to secure a suture 78. A lateral opening or eyelet 80 can be provided in the suture anchor tip 36 to receive a suture 82 or other medical device, such as suture tape, to be secured by the suture anchor 20. The suture 78 is threaded through open end 73 of the drive shaft 32 and progresses through the interior opening 62 of the drive shaft 32 to the handle 33 where it can be secured in a retention slot 71 (FIG. 13) or other retention structure. Maintaining force on the retention suture 78 as shown by arrows 110 (FIG. 22) will retain the suture tip tightly against the drive shaft 32.

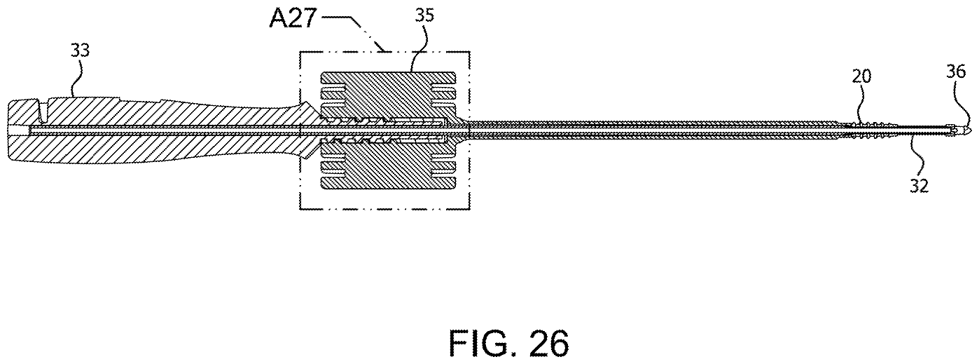

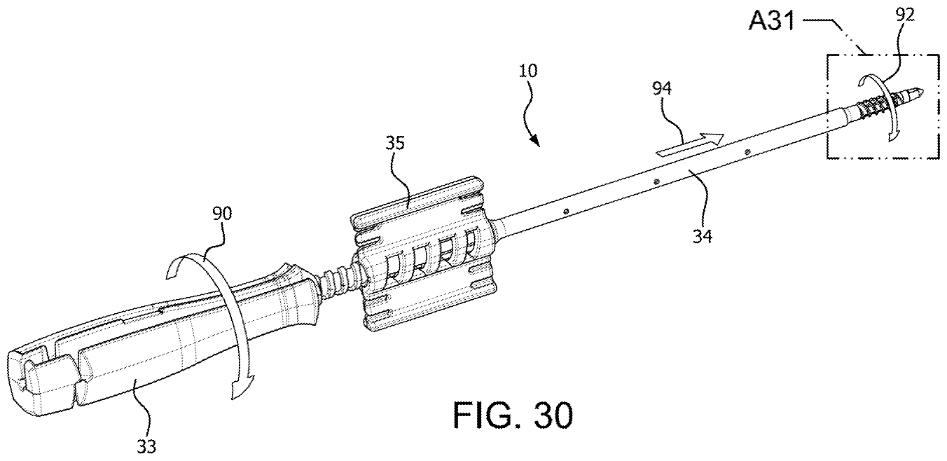

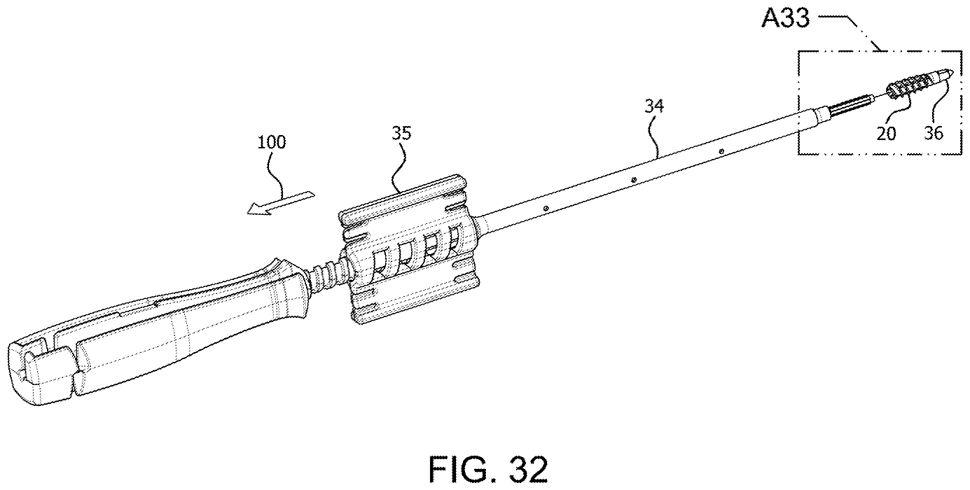

As shown in FIGS. 26-33, rotation of the handle 33 as shown by the arrow 90 rotates the drive shaft 32 and thereby the suture anchor 20 that is secured to the drive shaft 32, as indicated by arrow 92 (FIG. 30). Similarly, a forward pressure on the drive paddle 35 causes the drive sheath 34 to move forward in the direction shown by arrow 94 which will put pressure on the suture anchor 20 to drive the suture anchor 20 into the bone tunnel. Once positioned appropriately in the bone tunnel, the anchor insertion device 30 (FIG. 13) can be removed as shown by arrow 100 (FIG. 32) to leave the suture anchor 20 and suture anchor tip 36 in position in the bone tunnel.

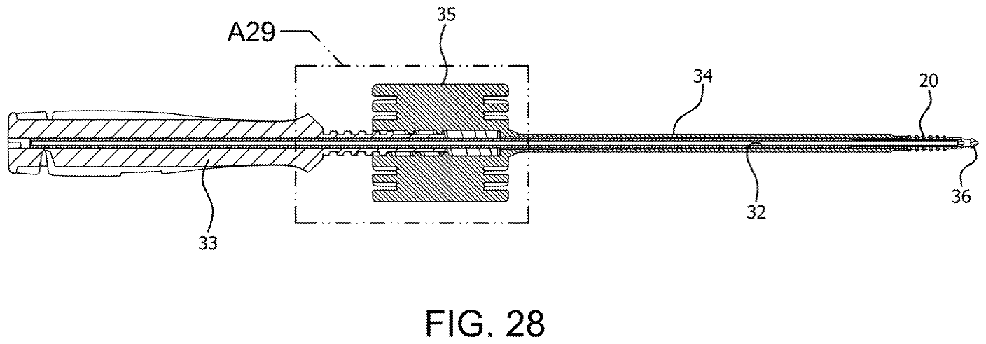

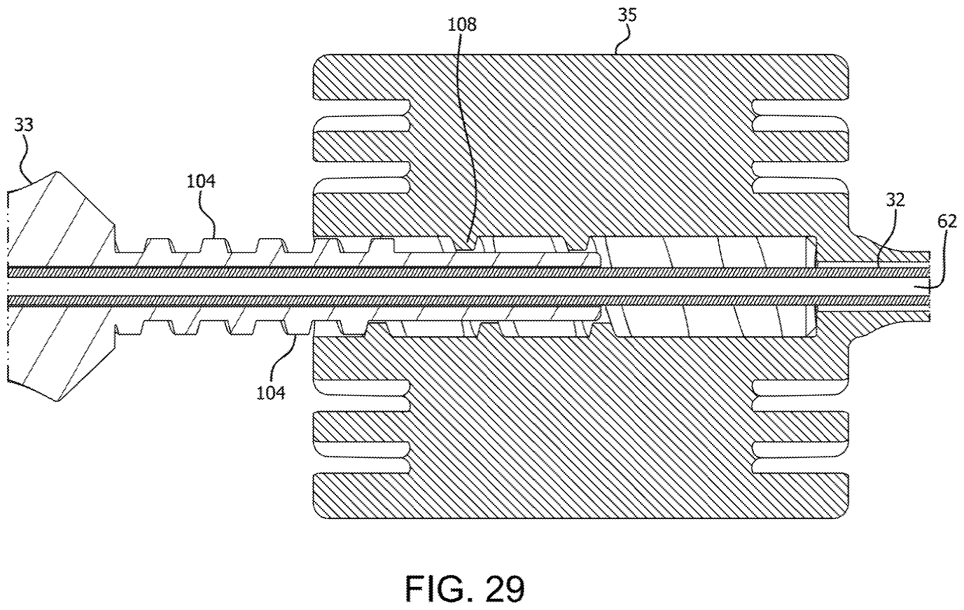

As shown in FIGS. 26-29, a distal end of the handle 33 has threads 104 which can engage similar cooperating threads 108 on the driver paddle 35. Continued rotation will cause relative movement between the drive paddle 35 and connected drive sheath 34 and the handle 33, as shown in FIGS. 28-29. This will have the effect of advancing the drive sheath 34 relative to the handle 33, and forcing the sheath 34 against the suture anchor 20. This force together with the rotation of the suture anchor 20 will thread the suture anchor 20 into the bone tunnel. As shown in FIG. 30, as the handle 33 is rotated as indicated by arrow 90, the anchor 20 will also rotate as indicated by arrow 92. Continued force on the drive paddle 35 will apply a force indicated by arrow 94 to drive the anchor into the bone tunnel as the anchor rotates. This will thread the suture anchor 20 into the bone tunnel.

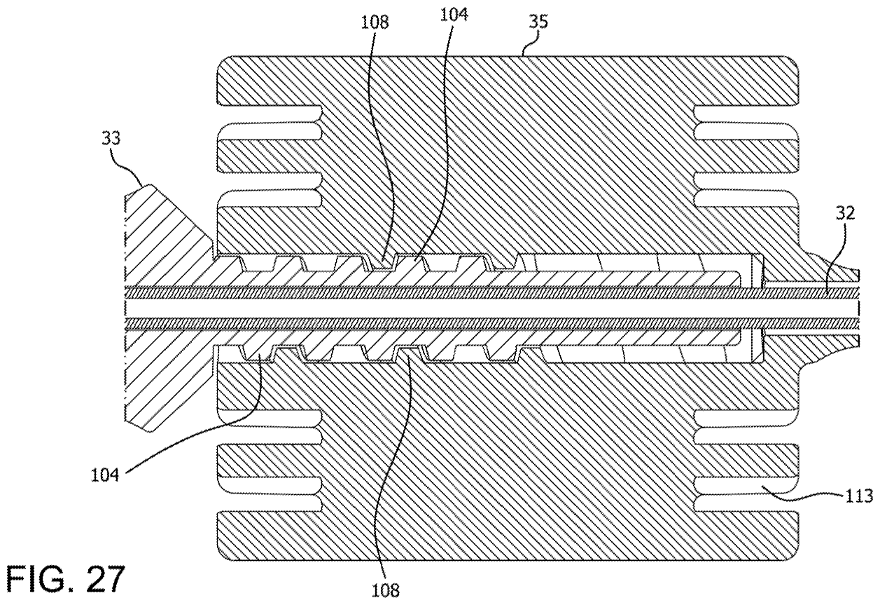

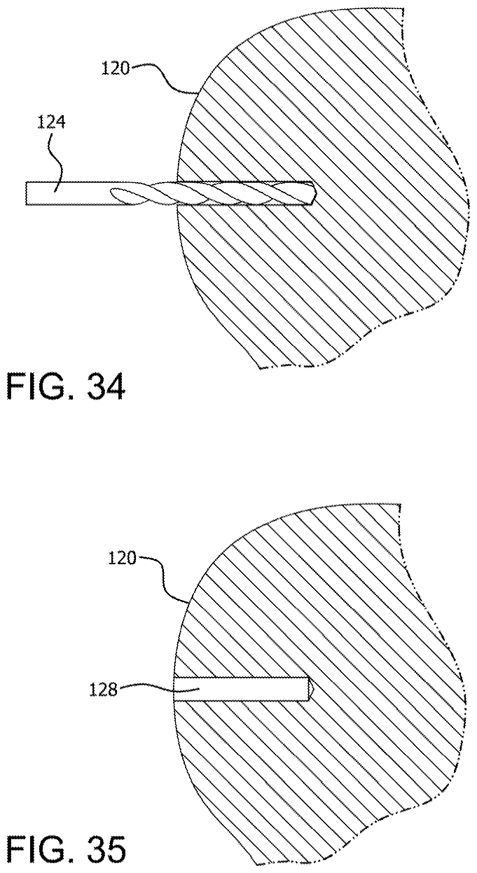

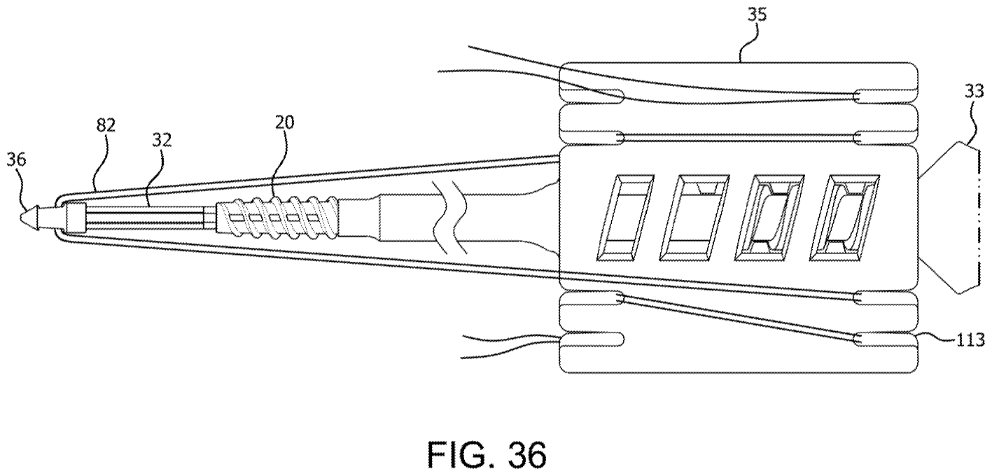

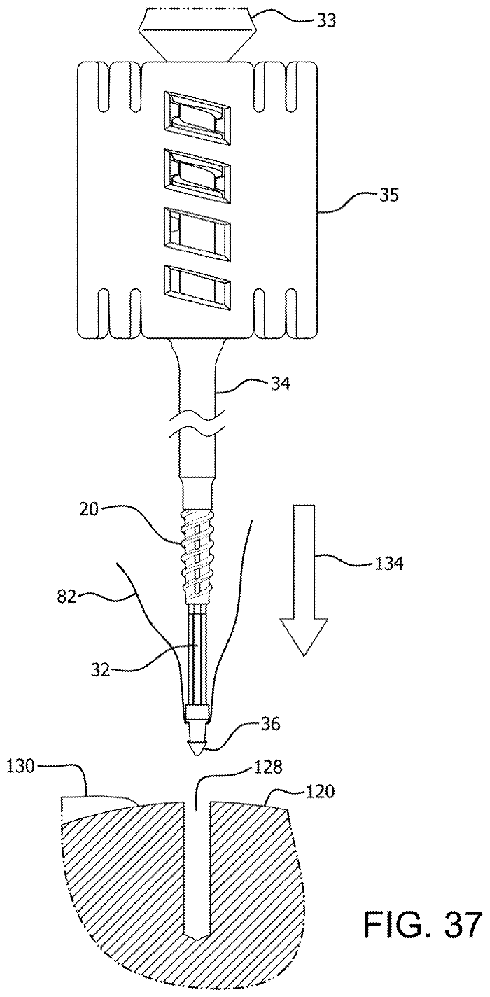

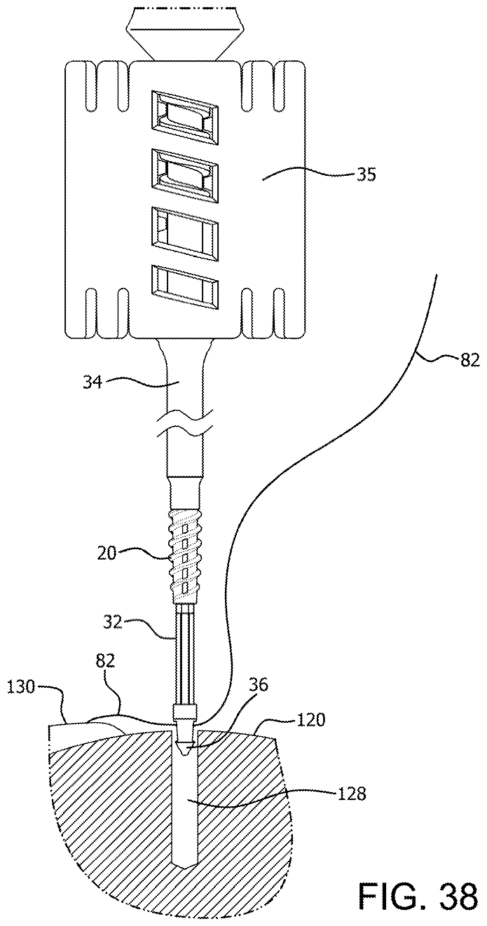

The operation of the suture anchoring system is shown in FIGS. 34-47. The surgeon initially makes a tunnel in the bone 120 using a drilling apparatus exemplified by drill bit 124 (FIG. 34). The suture 82 can be threaded through the lateral opening 80 and can be secured in slots 113 formed in the paddle 35 (FIG. 27). The bone tunnel 128 (FIG. 35) can be of differing sizes shapes and locations and will be determined by the surgeon according to the surgical plan. The suture anchoring system including the suture anchor 20, anchor tip 36, and suture 82 that is to be secured are introduced to the bone tunnel 128, as indicated by arrow 134 (FIGS. 36-37). In this example, the suture 82 will be used to attach tissue 130, however it will be appreciated that the suture 82 can be used according to a variety of procedures known in the art to secure differing tissues, organs and medical devices such as grafts, and the suture can be replaced by any medical device that is anchored by a suture anchor. The suture 82 is attached to the tissue 130. The suture anchor tip is then introduced into the bone tunnel 128. The drive shaft 32 and suture anchor tip 36 are advanced into the bone tunnel 128 until the suture anchor 20 is in contact with the bone 120. Tension is applied to the suture 82 as shown by arrow 138 as the suture anchor 20 is advanced (FIG. 39).

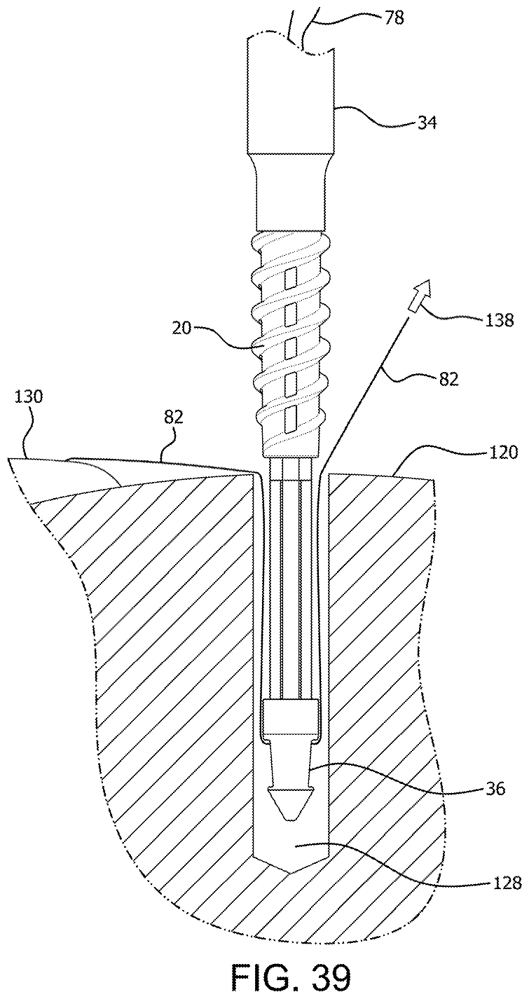

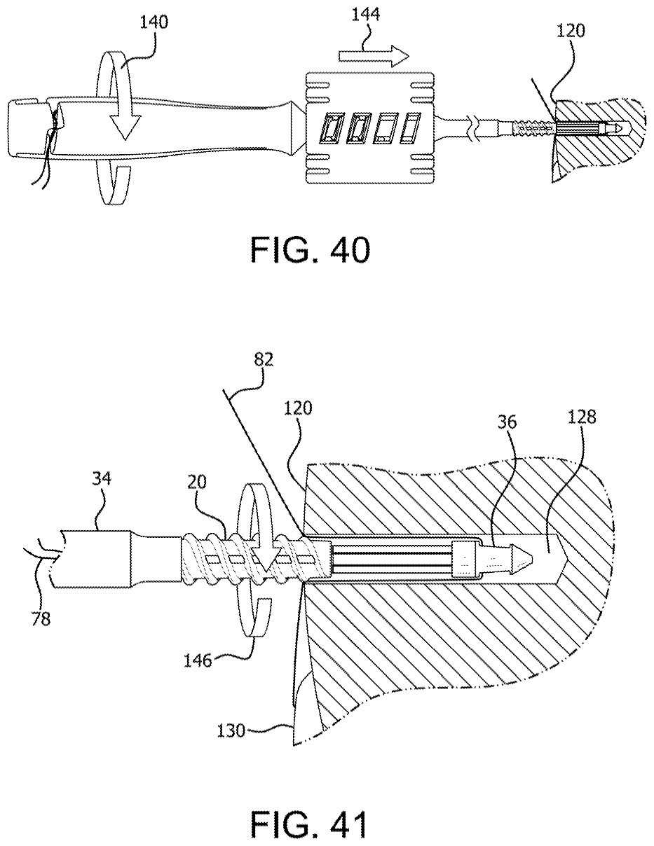

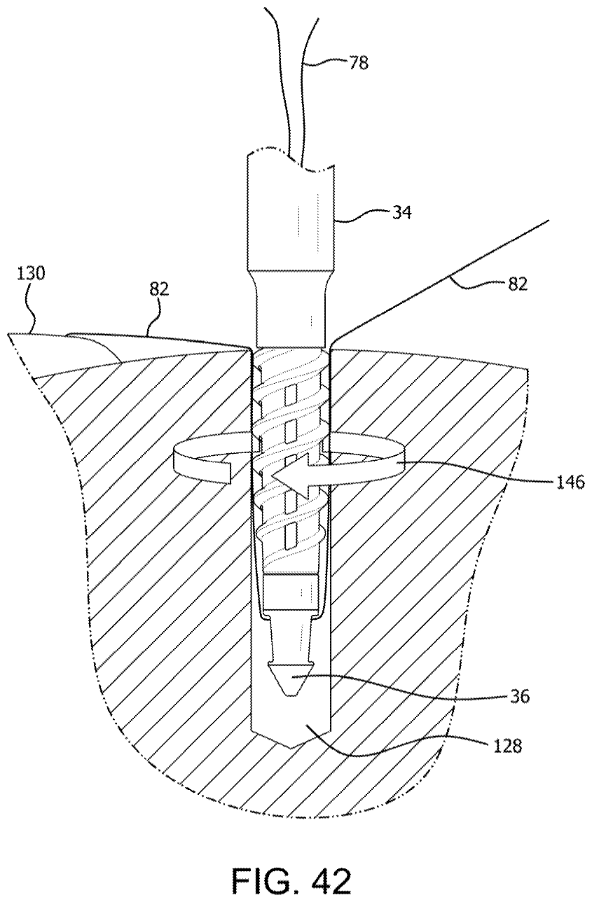

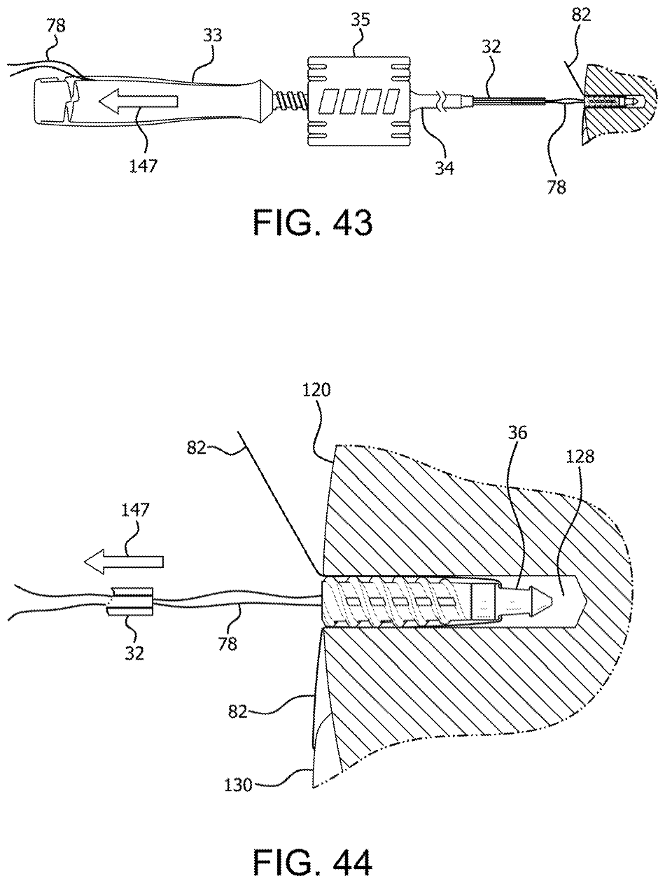

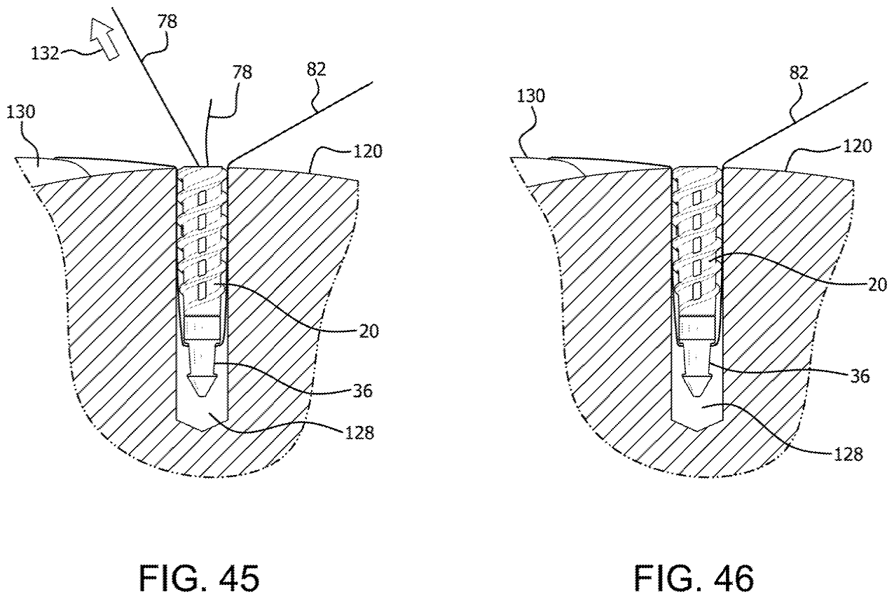

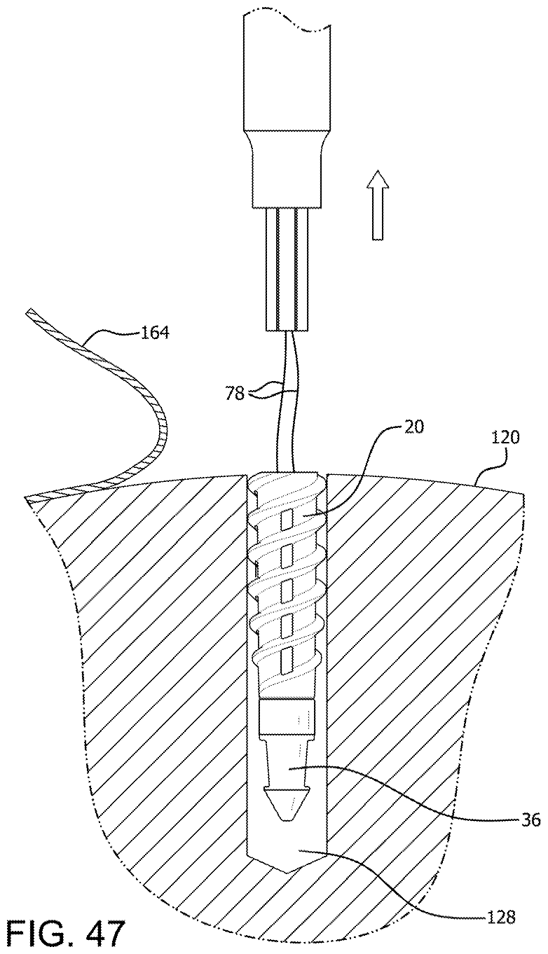

The handle 33 is then rotated in the manner shown by arrow 140 in FIG. 40. This will cause rotation of the suture anchor 20 as shown by arrow 146 (FIG. 41). A force indicated by arrow 144 advances the sheath 34 and by contact the suture anchor 20. Continued rotation will thread the suture anchor 20 into the bone tunnel 128, shown by FIG. 42. The retention suture 78 can be disconnected and the anchor insertion device 30 can then be withdrawn from the suture anchor 20 as shown by arrow 147 in FIGS. 43-44. The retention suture 78 can be tensioned as shown by arrow 132 in FIG. 45 and then tied and cut as shown in FIG. 46.

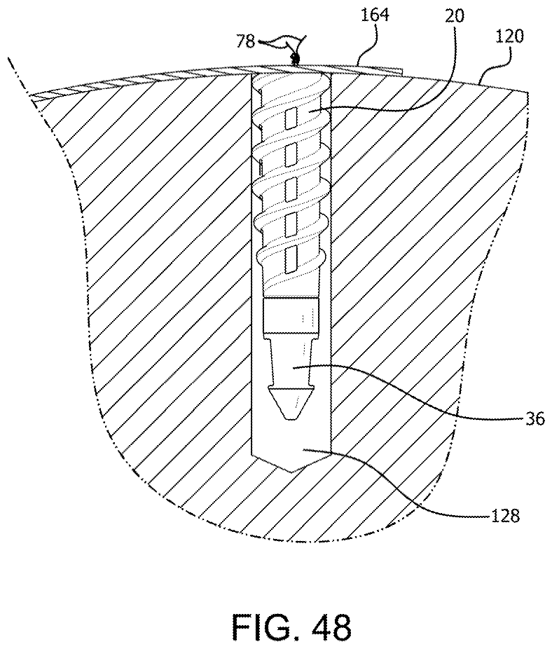

In some procedures it will be desirable that the retention suture 78 is used for the repair. This is shown in FIGS. 47-48. The suture 78 can be released from the drive shaft 32 and the handle 33 by untying the suture 78 from the handle and withdrawing the insertion device 30 from the suture 78, and then for example tying the suture 78 to secure a graft or tissue 164.

The invention as shown in the drawings and described in detail herein disclose arrangements of elements of particular construction and configuration for illustrating preferred embodiments of structure and method of operation of the present invention. It is to be understood however, that elements of different construction and configuration and other arrangements thereof, other than those illustrated and described may be employed in accordance with the spirit of the invention, and such changes, alternations and modifications as would occur to those skilled in the art are considered to be within the scope of this invention as broadly defined in the appended claims. In addition, it is to be understood that the phraseology and terminology employed herein are for the purpose of description and should not be regarded as limiting.

We claim:

1. A suture anchoring system, comprising
an anchor insertion device comprising a handle and a drive shaft with a long axis;
a suture anchor comprising an elongated anchor body having proximal and distal ends and a long axis, with an elongated axial drive opening extending along the long axis, the axial drive opening defining an inside surface of the anchor body, the anchor body having an outside surface radially outward from the inside surface; a plurality of axial drive grooves having a long axis parallel to the long axis of the elongated anchor body, the drive grooves having a radially inward and axially extending groove opening communicating with the axial drive opening, and a contact surface radially outward from the groove opening, the radially outward contact surface being of greater width than the groove opening, the axial drive grooves further comprising side surfaces extending between the contact surface and the axially extending opening of the axial drive grooves, the side surfaces being oriented at an acute angle to the radially outward contact surface, and threads on the outside surface of the anchor body;
wherein the drive shaft comprises radially outwardly extending axial drive projections configured to mate with the axial drive grooves.

2. The suture anchoring system of claim 1, wherein the axial drive grooves are circumferentially spaced apart and equidistant from adjacent axial drive grooves, and wherein the axial drive projections are circumferentially spaced apart and equidistant from adjacent axial drive projections, such that the drive shaft and the axial drive projections can be axially inserted into the elongated axial drive opening and the axial drive grooves.

3. The system of claim of claim 1, wherein the threads are provided as a double helix.

4. The system of claim 1, wherein in a lateral cross section orthogonal to the long axis of the anchor body, the axial drive grooves are isosceles trapezoidal in shape, and in a lateral cross section orthogonal to the long axis of the drive shaft, the axial drive projections are isosceles trapezoidal in shape.

5. The system of claim 1, wherein the threads are buttress threads.

6. The suture anchor of claim 1, wherein the contact surface is at least one selected from the group consisting of planar and curved.

7. The suture anchor of claim 6, wherein the contact surface is concave.

8. The suture anchor of claim 1, wherein the axial drive grooves extend from the proximal end to the distal end of the elongated anchor body.

9. The suture anchor of claim 1, further comprising friction projections on the inside surface of the axial drive opening.

10. The suture anchor of claim 1, wherein the anchor body further comprises a plurality of flow passages.

11. The suture anchor of claim 1, wherein the anchor body comprises elongated radially inwardly extending braces between the axial drive grooves.

12. The suture anchor of claim 11, wherein the anchor body further comprises a plurality of flow passages for permitting ingress of biological material from a patient into the axial drive opening, the flow passages passing through the braces.

13. The suture anchor of claim 1, wherein the anchor body comprises biocompatible plastic, the biocompatible plastics comprising at least one selected from the group of biocomposite and nonbiocomposite materials.

14. The suture anchor of claim 1, further comprising a suture retention tip comprising at least one eyelet for receiving a suture.

15. The suture anchor of claim 14, wherein the suture retention tip further comprises an open interior and a retention post in the open interior for engaging a retention suture.

16. The suture anchor of claim 15, wherein the suture retention tip is detachable from the anchor body.

17. A suture anchor, comprising:
an elongated anchor body having proximal and distal ends and a long axis, with an elongated axial drive opening extending along the long axis, the axial drive opening defining an inside surface of the anchor body, the anchor body having an outside surface radially outward from the inside surface;
a plurality of axial drive grooves having a long axis parallel to the long axis of the elongated anchor body, the drive grooves having a radially inward and axially extending groove opening communicating with the axial drive opening, and a contact surface radially outward from the groove opening, the radially outward contact surface being of greater width than the groove opening, the axial drive grooves further comprising side surfaces extending between the contact surface and the axially extending opening of the axial drive grooves, the side surfaces being oriented at an acute angle to the radially outward contact surface; and,
threads on the outside surface of the anchor body.

18. The suture anchor of claim 17, wherein the axial drive grooves are circumferentially spaced apart and equidistant from adjacent axial drive grooves.

19. The suture anchor of claim 17, wherein the threads are provided as a double helix.

20. The suture anchor of claim 17, wherein the threads are buttress threads.

21. The suture anchor of claim 17, wherein in a lateral cross section orthogonal to the long axis, the axial drive grooves are isosceles trapezoidal in shape.

22. A suture anchor comprising a suture anchor body having an external surface, and a double helix thread on the external surface.

23. The suture anchor of claim 22, wherein the double helix thread is a buttress thread.

24. The suture anchor of claim 22, wherein the suture anchor is made from a plastic material.

25. The suture anchor of claim 22, wherein the suture anchor comprises an open interior, and plurality of flow passages in the suture anchor body communicating with the open interior.
